(19) United States
(12) Patent Application Publication
(10) Pub. No.: US 2003/0134826 A1
(43) Pub. Date: Jul. 17, 2003

(54) Heterobifunctional cross-linking agent conjugate substituted by a bioactive group and a cell recognition unit

(76) Inventors: Karl-Heinz Glusenkamp, Marl (DE); Dagmar Dilloo, Dusseldorf (DE)

Correspondence Address: HESLIN ROTHENBERG FARLEY & MESITI PC, 5 COLUMBIA CIRCLE, ALBANY, NY, 12203, US

(21) Appl. No.: 10/298,855

(22) Filed: Nov. 18, 2002

Related U.S. Application Data
(63) Continuation of application No. PCT/EP01/05672, filed on May 17, 2001.

(30) Foreign Application Priority Data
May 17, 2000 (DE) DE 10024069.0

(51) Int. Cl. A61K 31/69; C07F 5/02

(52) U.S. Cl. 514/64; 548/405; 549/4; 562/7

(57) ABSTRACT

The present invention related to heterobifunctional cross-linkers substituted by a cell surface binding group or a cell recognising group, and also by a bio-active group, the bio-active group attached to a carbonyl of the cross-linker by an acid labile bond. Examples of suitable bio-active groups include an immunomodulator and an anti-neoplastic group. The conjugate enables, for example, the coupling of immunomodulators ex vivo to a target cell, for example a tumor antigen presenting cell. The conjugate can act as a non-toxic prodrug carrying a cytotoxin, for example, to a target tissue site, where the pH-dependency of the labile amide bond provides for cell selectivity of the masked cytotoxin. The invention further relates to a method for producing the cross-linkers, their use in treating disease, and ex-vivo cell labeling.

### RELATED APPLICATIONS

This application is a continuation under 35 U.S.C. §120 of International Application No. PCT/EP01/05672, which designated the United States and was filed on May 17, 2001, published in German on Nov. 22, 2001 as WO 01/87347, which claims priority from German Patent Application DE 10024069.0, filed on May 17, 2000. The entire teachings of the above applications are incorporated herein by reference.

### BACKGROUND OF THE INVENTION

A number of methods for treating tumours have been tested in animal models and in clinical studies. For example, Interleukin-2, known for its co-stimulating effects in activating immune cells, especially T-cells or NK-cells, has been systemically used to improve the immune status of a tumour patient. However, due to its effects on non-mutated cells, Interleukin-2 causes considerable systemic side effects.

To develop a more specific therapy based on the immunomodulating effects of cytokines, attempts have been made to transfect tumour cells with cytokines, including IL-2. In vivo, such transfected tumour cells were supposed to ensure a tumour-specific T-cell response through increased local IL-2 expression in the immediate vicinity of the tumour antigen. Indeed, in tests with mice, significant prolongation of survival time as well as an expansion and activation of tumour-specific T-cells was achieved by manipulating tumour cells ex vivo by using gene technology procedures, for example, by using suitable viral vectors to express a combination of IL-2 and lymphotoxin (to increase the local concentration of immune cells). In the literature, this therapeutic method is referred to as tumour vaccination. However, a disadvantage of vectors currently admitted for clinical application is that many primary human tumour cells can be transfected with only low efficiency. Furthermore, the long-term side effects of those clinical viral vectors are not predictable, and preparation of such viral vectors is expensive.

In order to achieve delayed release of cytokines when fighting tumours, cytokines in capsule form were introduced. The shell of the capsules has a composition which only under certain physiological conditions, releases the capsule contents. Such capsules were mixed with tumour cells, and this mixture injected subcutaneously as a tumour vaccine. This method, however, does not guarantee that there is a synergism—which is responsible for the effect of the tumour vaccines—between the tumour cell presenting the antigen, and the capsule containing the cytokine. Also, the synergism requires that the two components be in close vicinity.

Thus, there is a need for a vaccine system that ensures close vicinity of the components.

In another approach, cytokines were coupled with tumour-specific antibodies. Such complexes or cytotoxic substances, bound to tumour-specific antibodies, were systemically administered to tumour patients, in the hope that the complexes would recognise tumour cells, especially metastasised cells, and that the tumour cells would be specifically attacked in this manner. However, in the body of the patient it is difficult to achieve a specific, intensive enrichment in the tumour area, with the result that patients could experience considerable side effects caused by these complexes. In addition, when directly coupling the effective agent to the antibody recognising the cell, a delayed release of, eg., cytokines, which have paracrine effects in the area of the tumour cells presenting the tumour antigens, is not ensured.

In order to release agents in active form only under certain physiological conditions, compounds which have a labile component depending on acidity are disclosed in DE 43 26 273 A1, the teachings of which are incorporated herein by reference in their entireties. The conjugate, an agent coupled with the acid labile component, has the character of a so-called “prodrug.” It is preferably used at low pH values.

In the conjugates disclosed in DE 43 26 273 A1, the masking of biologically active substances is achieved by special bicyclic carbon acid hydrides, wherein innovative agents (prodrugs) having the following general formulas (Ia) or (IIa) emerge:

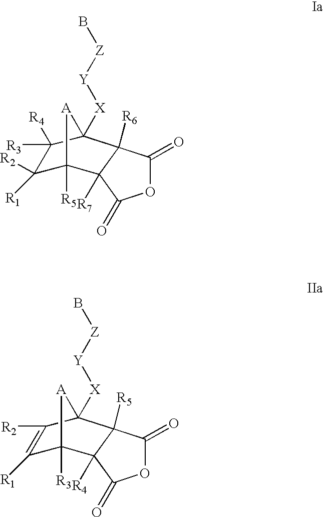

wherein R¹ to R⁴, or R¹ and R² respectively are identical or different and represent hydrogen, substituted cycloalkyl having 3 to 6 carbon atoms or linear chain or branched alkyl having up to 8 carbon atoms, or substituted phenyl; or wherein R¹ to R⁴, or R¹ and R² together form either an unsaturated, 5- to 7-segment heterocycle having up to 3 heteroatoms of the N, S or 0 series, or a 4- to 7-segment carbocycle; or wherein R¹ and R³, or R² and R⁴ together form a 3-segment heterocycle with 0 or NR¹⁰; R¹⁰ is hydrogen or a linear chain or branched alkyl of 1 to 6 carbon atoms;

X is an oxygen or sulfur atom, or a group of the formula —CR¹¹R¹²—; R¹¹ and R¹² are identical or different, and are selected from hydrogen, substituted cycloalkyl of 3 to 6 carbon atoms, a linear chain or branched alkyl having 1 to 8 carbon atoms, substituted phenyl, imidazolyl, and indolyl;

Y depends on the particular identity of X, and is an oxygen or sulfur atom or a group of the formula —CO—, —CR¹³R¹⁴, —NR¹³; R¹³, R¹⁴, R¹⁵ are each independently hydrogen, a linear chain or branched alkyl having 1 to 6 carbon atoms, or phenyl;

Z depends on the respective meaning of Y, and is selected from a direct bond, an oxygen or sulfur atom, a group of the formula CO—, —CS—, —SO₂—, —CO—NR¹⁶, —CS—NR¹⁷—, P(O)(OR¹⁸)₂, —S— or CO—CH₂—CH₂—S—S—; R¹⁶, R¹⁷, R¹⁸ are each independently hydrogen, linear chain or branched alkyl having 1 to 6 carbon atoms, or phenyl;

B depends on the particular identity of Z, and is substituted cycloalkyl of 3 to 6 carbon atoms, linear chain or branched alkyl of 1 to 8 carbon atoms, substituted phenyl imidazolyl, indolyl, thiomethyl, thioethyl, 2-pyridyl, or adamantyl;

R⁵ has the above-mentioned meaning of R¹ to R⁴, is identical to them or different, or can have the same meaning as X, Y, Z and B;

A is an oxygen or sulfur atom, or represents the remainder of the formula NR¹⁹; R¹⁹ is hydrogen, linear chain or branched alkyl having 1 to 8 carbon atoms, or represents the remainder of the formula —CR²²R²³—; R²⁰ and R²¹ are each independently hydrogen, linear chain or branched alkyl having 1 to 6 carbon atoms, or represents the remainder of the formula CR²²R²³—Cr²⁴R²⁵; R²², R², R²⁴, and R²⁵ are the same or different, and are each independently hydrogen or alkyl having up to 6 carbon atoms;

R⁶ and R⁷ are the same or different, and are each independently hydrogen, a linear chain or branched alkyl of 1 to 8 carbon atoms, nitro, halogen, carboxy, or cyano;

G is hydroxy, an amino acid bound via a primary or secondary amino group and their monomer or polymer derivatives, such as tryptophan, tryptamine, N-Me-tryptophan, N-Me-tryptamine, ditryptophan, tryptophanmethylester, or cytotoxic compounds containing amino groups, such as melpahlan, Nor-N-lost, cis-platinum or carboplatinum, or other platinum complexes, anthracycline, such as daunomycin, or also mitomycin C, and the various bleocimines; or analgetic, antibiotic, anaesthetic, antiphlogistic, antiseptic, antimycotic or antiviral agents, which are known in relevant literature, having a primary or secondary amino group; and

F has the above-described meaning of G, with the proviso that one of the substituents F or G are hydroxy.

The compounds disclosed in DE 43 26 273 A1 are obtained by adding the agent containing a primary or secondary amino group to the bicyclic acid anhydride (I) or (II). Thus, the bicyclic acid anhydride represents a masking substance. The bond between the agent and masking substance is a labile amide bond, the stability of which can be influenced by specific chemical modification in the side group —X—Y-Z-B. Furthermore, the pH-value has a large influence on the stability of the labile amide bond. In the pH-range from 7.0 to 7.5, the compounds are of high stability, but they are substantially more labile around pH 6.4. The compounds or conjugates represent “prodrugs,” non-toxic predecessor compounds of common drug substances, especially cytotoxins.

Such pH-labile prodrugs are especially beneficial in the treatment of neoplastic diseases. Indeed, the pH-dependency of the labile amide bond provides the masked cytotoxin a greater selectivity for cancer cells, since the pH-value in neoplastic tissue lies around an average value of 6.8, against a pH of 7.1 in non-neoplastic or normal tissue. This difference is significantly increased by stimulation of the aerobic glycolysis of malignant cells by systemic supply of glucose (average pH 6.4), whereas the measured pH distribution in normal tissue varies only slightly. Thus, the pH-dependency causes a location-specific and thus delayed release of the active form from the prodrug. Thus, the masking substance used for masking the active component is also called a slow-release component.

The acid labile slow-release components in conjugates, disclosed in DE 43 26 273 A1, mask conventional cytotoxins, for example, in the conjugates in the form of non-toxic predecessor compounds which develop their cell toxicity only after splitting the labile amide. These conjugates, however, are not suitable for vaccination against neoplastic disease.

Furthermore, boronic acid derivatives of the formula

Z-Y—B(OH)₂

are known from DE 196 45 601 A1, where Y is a substituted or unsubstituted alkylene of the saturated unbranched or branched, or the unsaturated type, preferably propylene or 2-methyltrimethylene, or a substituted cyclic residue of the saturated heterocyclic, alicyclic or aromatic type, especially arylene, such as 1,3-phenylene or benzylene, and where Z is a substituted or unsubstituted bio-specific compound.

Bio-specific compounds are disclosed in DE 196 45 601 A1. Bio-specific compound, as the term is used herein, is a compound that is either biologically effective, pharmaceutically effective, biochemically effective, or diagnostically effective. A bio-specific compound especially preferred in this document, is a colouring substance which fluoresces depending on the pH-value, preferably a substituted fluorescein. With these boronic acid derivatives, however, the bond between bio-specific compound Z and the residual —Y—B(OH)₂ is stable.

These boronic acids can react with vicinal OH groups in a variety of substrates, forming a cyclic ester, which is unusually stable at physiological pH-values. Cells contain a plurality of polymer carbohydrates having free, vicinal diol groups, and they can thus be specifically marked with boronic acids. The boronic acid derivatives described in DE 196 45 601 A1 disclose the marking of cells with conjugates, but these markings are not suitable for specific therapeutic use in cancer patients, especially for vaccination against neoplastic disease.

Thus a need exists for new compositions and methods for production of such compositions, that can be used to produce effective cancer vaccines in a cost-effective manner, while at the same time fulfilling the requirements for optimal patient safety.

### SUMMARY

The present invention relates to heterobifunctional cross-linkers substituted by a cell recognition unit and also by a bio-active group, such as an immunomodulator or an anti-neoplastic group. It further relates to a method for producing these cross-linkers and their use in ex-vivo cell labeling and cells labeled in such manner.

Accordingly, the present invention discloses conjugates that have at least one bioactive group, such as, for example, an immunomodulator or an anti-neoplastic group with a labile bond, and also have a cell surface binding component that is capable of specifically interacting with cell surfaces or cell parts by means of specific or unspecific interaction, wherein a controlled release rate of at least one immunomodulator or anti-neoplastic group, which has a labile bond in the conjugate, is to be achieved at the location of the interaction reaction.

In a preferred embodiment of the invention, a compound has Formula I or II:

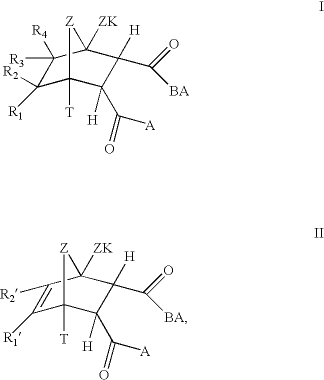

wherein BA is a bio-active group attached to carbonyl by an acid labile bond;

ZK is a cell surface binding group or a cell recognising group;

R₂ and R₄ are hydrogen or together form —O— or —NR₁₀—, wherein R₁₀ is a linear chain or branched alkyl of 1 to 6 carbon atoms; R₁, R₃, R′₁ and R′₂ are each chosen independently from hydrogen, alkyl, and substituted alkyl; or R₁ and R₃ together, or R′₁ and R′₂ together form a 5 to 7 segment unsaturated heterocycle of 1 to 3 heteroatoms selected from N—, S—, and O; or R₁ and R₃ together, or R′₁ and R′₂ together form a 4 to 6 segment carbocycle; Z is chosen from oxygen, sulfur, —NR₅—, —CR₆R₇—, and —CR₆R₇—CR₈R₉—, wherein R₅ is H or C₁₋₈-alkyl, and R₆, R₇, R₈ and R₉ are each independently H or C₁₋₆-alkyl; A is hydroxy; and T is chosen from the group consisting of a cell surface binding group, a cell recognising group, hydrogen, alkyl, and substituted alkyl.

In one embodiment, R₁, R₃, R′₁ and R′₂ are each intended to include, independently, hydrogen or a linear, branched or cyclic, optionally substituted alkyl group having 1 to 8 carbon atoms, wherein the substituents are preferably selected from halogen, amino, cyano, carboxy, linear chain or branched alkoxy having 1 to 6 carbon atoms, and hydroxy.

According to an embodiment, T independently is as defined for ZK, or is H, a linear, branched or cyclic, optionally substituted alkyl group of 3 to 8 carbon atoms, wherein the substituents are preferably halogen, amino, cyano, carboxy or sugar residue.

The invention also relates to a compound of Formula I or II:

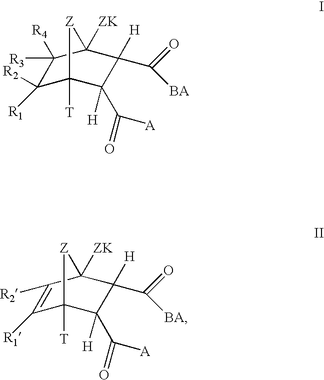

wherein BA is an immunomodulator group or an anti-tumor group attached to carbonyl by an acid labile bond; ZK is a cell surface binding group or a cell recognising group that is capable of specific or unspecific cell recognition; R₂ and R₄ are hydrogen or together form —O— or —NR₁₀—, wherein R₁₀ is a linear chain or branched alkyl of 1 to 6 carbon atoms; R₁, R₃, R′₁ and R′₂ are each chosen independently from hydrogen, alkyl, and substituted alkyl; or

R₁ and R₃ together, or R′₁ and R′₂ together form a 5 to 7 segment unsaturated heterocycle of 1 to 3 heteroatoms chosen from N—, S—, and O; or

R₁ and R₃ together, or R′¹ and R′₂ together form a 4 to 6 segment carbocycle;

Z is chosen from oxygen, sulfur, —NR₅—, —CR₆R₇—, and —CR₆R₇—CR₈R₉—, wherein R₅ is H or C₁₋₈-alkyl, and R₆, R₇, R₈ and R₉ are each independently H or C₁₋₆-alkyl;

A is hydroxy; and T is a cell surface binding group, a cell recognising group, hydrogen, alkyl, or substituted alky. The acid labile bond by which BA is attached to carbonyl is labile especially at pH-values≦6.8.

The invention also provides a pharmaceutical composition including a pharmaceutically acceptable carrier and a compound of Formula I or II as described herein.

In another embodiment, the invention relates to a method for treating a neoplastic disease in a patient in need thereof including administering to the patient a therapeutically effective amount of a compound of Formula I or Formula II described above.

The invention also includes a method for treating an autoimmune disease or an infectious disease in a patient in need thereof, or inhibiting the onset of symptoms of the autoimmune disease or the infectious disease, including administering to the patient a therapeutically effective amount of a compound of Formula I or Formula II described above.

In yet another embodiment, the invention includes a method for producing a compound according to claim 1 of the Formula I or II:

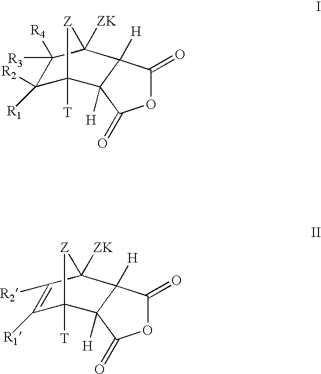

wherein R₁, R₃, R′₁, R′₂, A, T, Z, ZK and BA are as defined in claim 1; and

R₂ and R₄ are H, the method including the steps of reacting a diene of formula III:

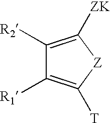

wherein R′₁, R′₂, T, Z and ZK are as defined in claim 1, with maleic acid anhydride, thereby forming a product of formula IV:

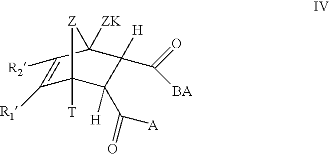

wherein R′₁, R′₂, T, Z and ZK are as defined in claim 1; optionally reducing the product of formula IV, thereby forming a product of formula V:

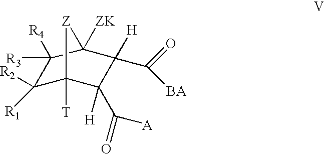

wherein R₁, R₃, T, Z and ZK are as defined in claim 1; and R₂ and R₄ are H; and

incubating the product of formulas IV or V with a bio-specific compound containing a primary or secondary amine group, thereby obtaining a product of formula I or II.

The subject-matter of the present invention provides many advantages. For example, the structure of a conjugate according to the invention enables the coupling of a bioactive agent such as an immunomodulators ex vivo to a target cell, especially a cell presenting a tumour antigen, in such a manner that a preferably successive paracrine release of the same can occur in vivo in close vicinity to the tumour antigen under respective acidic conditions of the individual physiological environment. Once the two immunological activator signals (tumour antigen and immune stimulant) meet, activation of the immune system, i.e. especially the immune cells, can proceed effectively.

### DETAILED DESCRIPTION

A description of the preferred embodiments of the invention follows. The features and other details of the compounds and methods of the invention will now be more particularly described and pointed out in the claims. It will be understood that particular embodiments of the invention are shown by way of illustration and not as limitations of the invention. At the outset, the invention is described in its broadest overall aspects, with a more detailed description following.

The present invention discloses conjugates that have at least one bioactive group, such as, for example, an immunomodulator or an anti-neoplastic group with a labile bond, and also have a component that is capable of specifically interacting with cell surfaces or cell parts by means of specific or unspecific interaction, wherein a controlled release rate of at least one immunomodulator or anti-neoplastic group, which has a labile bond in the conjugate, is to be achieved at the location of the interaction reaction.

In a preferred embodiment of the invention, a compound has Formula I or II:

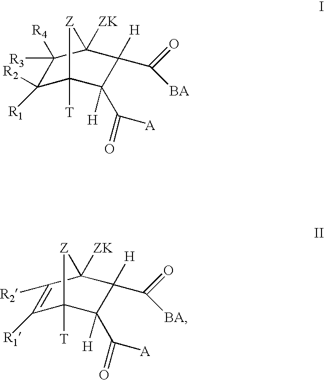

wherein BA is a bio-active group attached to carbonyl by an acid labile bond;

ZK is a cell surface binding group or a cell recognising group; R₂ and R₄ are hydrogen or together form —O— or —NR₁₀—, wherein R₁₀ is a linear chain or branched alkyl of 1 to 6 carbon atoms; R₁, R₃, R′₁ and R′₂ are each chosen independently from hydrogen, alkyl, and substituted alkyl; or R₁ and R₃ together, or R′₁ and R′₂ together form a 5 to 7 segment unsaturated heterocycle of 1 to 3 heteroatoms selected from N—, S—, and O; or R₁ and R₃ together, or R′₁ and R′₂ together form a 4 to 6 segment carbocycle; Z is chosen from oxygen, sulfur, —NR₅—, —CR₆R₇—, and —CR₆R₇—CR₈R₉—, wherein R₅ is H or C₁₋₈-alkyl, and R₆, R₇, Rg and Rg are each independently H or C₁₋₆-alkyl; A is hydroxy; and T is chosen from a cell surface binding group, a cell recognising group, hydrogen, alkyl, and substituted alkyl.

In one embodiment, R₁, R₃, R′₁ and R′₂ are each intended to include, independently, hydrogen or a linear, branched or cyclic, optionally substituted alkyl group having 1 to 8 carbon atoms, wherein the substituents are preferably selected from halogen, amino, cyano, carboxy, linear chain or branched alkoxy having 1 to 6 carbon atoms, and hydroxy.

According to an embodiment, T independently is as defined for ZK, or is H, a linear, branched or cyclic, optionally substituted alkyl group of 3 to 8 carbon atoms, wherein the substituents are preferably halogen, amino, cyano, carboxy or sugar residue.

As the termas are used herein, alkyl and alkane are intended to include linear, branched, or cyclic hydrocarbon structures and combinations thereof. Lower alkyl refers to alkyl groups of from 1 to 6 carbon atoms. Examples of lower alkyl groups include methyl, ethyl, propyl, isopropyl, butyl, s-and t-butyl and the like. Preferred alkyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl and the like.

(C₁ to Cₙ) Hydrocarbon includes alkyl, cycloalkyl, alkenyl, alkynyl, aryl and combinations thereof containing only hydrogen and one to n carbons. Examples include vinyl, allyl, cyclopropyl, propargyl, phenethyl, cyclohexylmethyl, camphoryl and naphthylethyl. Saturated (C₁ to Cₙ) hydrocarbon is identical in meaning to (C_{1 to C}ₙ) alkyl or (C₁ to Cₙ) alkane as the terms are used herein.

Alkoxy or alkoxyl refers to groups of from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, 196, but without the restriction of 127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively. Examples include ethylaminoethyl and methylthiopropyl.

Acyl refers to groups of from 1 to 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons.

Aryl and heteroaryl mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. As commonly understood, when referring to aryl as a substituent, it is intended that the point of attachment is a ring carbon of the aryl group (or ring carbon or heteroatom of the heteroaryl). For the purpose of the present invention, aryl and heteroaryl refer to systems in which at least one ring, but not necessarily all rings, are fully aromatic. Thus aromatic 6- to 14-membered carbocyclic rings include, e.g., benzene, naphthalene, indane, tetralin, benzocycloheptane and fluorene and the 5- to 10-membered aromatic heterocyclic rings include, e.g., imidazole, pyridine, indole, isoindoline, thiophene, benzopyranone, thiazole, furan, benzimidazole, quinoline, isoquinoline, tetrahydroisoquinoline, quinoxaline, tetrahydrocarboline, pyrimidine, pyrazine, tetrazole and pyrazole.

Alkylaryl means an alkyl residue attached to an aryl ring. As commonly understood, when referring to alkylaryl as a substituent, it is intended that the point of attachment is the alkyl group. Examples of C1 C3 alkylaryl are benzyl, phenethyl, phenylpropyl and naphthylethyl. Alkylheteroaryl means an alkyl residue attached to a heteroaryl ring. Examples include, e.g., pyridinylmethyl, pyrimidinylethyl and the like.

Heterocycle means a cycloalkyl or aryl residue in which from one to three carbons is replaced by a heteroatom selected from the group consisting of N, 0 and S. The nitrogen and sulfur heteroatoms may optionally be oxidized, and the nitrogen heteroatom may optionally be quaternized. It is to be noted that heteroaryl is a subset of heterocycle in which the heterocycle is aromatic. Examples of heterocyclyl residues additionally include piperazinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

Substituted alkyl, aryl, cycloalkyl, heterocyclyl etc. refer to alkyl, aryl, cycloalkyl, or heterocyclyl wherein up to three H atoms in each residue are replaced with loweralkyl, halogen, haloalkyl, hydroxy, hydroxymethyl, loweralkoxy, perfluoroloweralkoxy, carboxy, carboalkoxy (also referred to as alkoxycarbonyl), carboxamido (also referred to as alkylaminocarbonyl), sulfonamido, aminosulfonyl, alkylaminosulfonyl, cyano, carbonyl, nitro, amino, alkylamino, dialkylamino, ureido, alkylureido, mercapto, alkylthio, sulfoxide, sulfone, acylamino, amidino, phenyl, benzyl, heteroaryl, phenoxy, benzyloxy, or heteroaryloxy.

The term “halogen” means fluorine, chlorine, bromine or iodine. As used herein, the terms “treatment” or “treating” a disease in a patient are intended to include prophylaxis. The terms include amelioration, prevention and relief from the symptoms and/or effects associated with these disorders. The terms “preventing” or “prevention” refer to administering a medicament beforehand to forestall or obtund an attack. Persons of ordinary skill in the medical art (to which the present method claims are directed) recognize that the term “prevent” is not an absolute term. In the medical art it is understood to refer to the prophylactic administration of a drug to diminish the likelihood or seriousness of a condition, and this is the sense intended.

As the term is used herein, “bioactive group” means any chemical species having an effect, either direct or indirect, on a living system, such as, for example, a cell, tissue, or organism. Example of bioactive groups include pharmaceutically active groups and therapeutic substances. As used herein, the terms “therapeutically effective substance” or “therapeutic substance” include:

(i) Compounds and compositions recognized in the official United States Pharmacopoeia, the official Homeopathic Pharmacopoeia of the United States, or the official National Formulary, or any supplement of any of them;

(ii) Compounds and compositions intended for use in the diagnosis, cure, mitigation, treatment, or prevention of disease in man or other animals; and

(iii) Compounds and compositions (other than food) intended to affect the structure or any function of the body of man or other animals. Many other examples of drugs and genetic material are well known, including drugs such as anaesthetics, heparin, erythropoietin, growth hormone, steroids, various peptides, and genetic material such as large DNA segments, RNA, small antisense oligonucleotides, and immunological material generally, including vaccines and adjuvants.

As the terms are used herein, “cell recognising group,” “cell surface binding group,” and “cell surface binding function,” mean molecules which have the property to bind to the cell surface either specifically (e.g., receptor/ligand, antibody directed to a cell surface protein) or unspecifically because of its “sticky” character (like aryl boron acid). “Sticky character” or “stickiness,” as the terms are used herein, means that the molecule so described adheres, attaches, binds, or sticks to a cell surface or cell surface molecule to a greater extent than does a molecule that lacks a sticky character. The “cell recognising unit” or “cell surface binding group,” is that component of the inventive molecule that allows the three-component inventive molecule to bind to the cell surface, according to the definition given above for the “cell surface binding function.”

The cell recognising unit (ZK) of a conjugate according to the invention can be of a specific or unspecific nature. If it is of specific nature, it will preferably be based on a receptor/ligand interaction. Here, the cell recognising unit in a conjugate according to the invention can be both a receptor recognising a membrane-residing ligand on the target cell, or a ligand interacting with a membrane-residing receptor on the target cell. Thus for example, in a conjugate according to the invention, the conjugate including a ZK, can preferably contain a soluble Fas-ligand which docks onto a Fas-receptor on a target cell, or, conversely, it can be a Fas-receptor, preferably a Fas-receptor without trans-membrane and intracellular sections, which facilitates binding to a membrane-residing Fas-ligand of a target cell when contact is made between the conjugate according to the invention and the target cell.

In this manner, the respective binding partners of all receptors or ligands, which physiologically occur on the membrane of the target cell as extracellular, can be used as a ZK in a conjugate according to the invention. By transfection with DNA that codes for certain, typically membrane-residing proteins not being expressed physiologically on the target cells, or at a low rate only, the target cells can possibly be altered so that the binding partners of the transfected protein may also be considered as a ZK in a conjugate according to the invention. The biological ligands or receptors used preferably as a ZK in a conjugate according to the invention for coupling to target cells, will have a particularly preferred recombinant form wherein the amino acid sequence is truncated to the binding domain or domains.

Immunomodulators are those substances which are able to modulate the immunesystem either by suppressing the immunesystem or by enhancing the immune response. An immunomodulator is a substance that has a regulator function for the immune system. The regulator function can be, for example, a stimulating function, an immunizing function, or a suppressant function. Some examples of immunomodulators include cytokines and other naturally occuring proteins, antigens and antigen fragments, small organic drugs, and phytopharmaceuticals. Other examples are provided below.

As the terms are used herein, “neoplastic,” “cancer,” “tumour,” and “tumor” have the same meaning and refer to the new growth of tissue having no physiological function.

Preferably, a conjugate according to the invention can be multimerised, in particular dimerised, trimerised, tetramerised or pentamerised, for example, by covalent and/or non-covalent interactions, and can be correspondingly present as a dimer, trimer, tetramer or pentamer. Multimerisation can occur via linking of immunomodulators, heterobifunctional cross-linkers, or, preferably, via the ZK. Here, a conjugate according to the invention can be designed in a way that only one ZK, ZK1, has an immunomodulator via an acid-labile cross-linker, whereas the other cell recognition units dimerise, trimerise, tetramerise or pentamerise with ZK1; however they do not have any immunomodulators or cross-linkers. Alternatively, however, according to the invention, dimers, trimers, tetramers or pentamers of conjugates according to the invention can also be present, each having a cross-linker and immunomodulator, where dimerising or multimerising occurs preferably via the respective ZK, possibly also via the immunomodulators combined in the dimer or multimer. By dimerisation or multimerisation, the affinity and/or avidity of conjugates according to the invention to the target cells can be increased. Also, dimerisation or multimerisation can serve to combine conjugates according to the invention with different immunomodulators into a dimerised or multimerised complex.

Two conjugates according to the invention, having at least one ZK and an antibody which, for example, recognises a certain group on the cross-linker or the ZK, may also be combined to form a dimer of a conjugate according to the invention. As a result of this method, cross-linked conjugate dimers can be coupled onto a target cell surface via antibodies.

When choosing the ZK responsible for cell recognition, it must be considered that for certain applications, in particular for conventional vaccination against neoplastic disease, that the membrane-residing binding partner, after binding of ZK in the conjugate according to the invention, is not internalised into the cell. Thus, when using the conjugate for cancer vaccination, receptor ligand systems have to be chosen which ensure that an incorporation of the complex does not occur, which would enable a release of the immunomodulator into the extracellular space.

Alternatively, however, it may be desirable, eg. in vaccination against neoplastic disease, that the immunomodulator is internalised into the cell. In this case, according to the invention, the ZK is chosen in a manner that after binding of a conjugate according to the invention to the binding partner onto the target cell surface, an incorporation of the conjugate receptor complex occurs, releasing the immunomodulator into the intracellular space. Typically, in this case, the immunomodulator will be a protein which acts as gene activator for, for example, cytokines or other immunologically relevant proteins, eg. by direct or indirect binding to regulatory units on the DNA or so-called enhancer areas.

Preferably, antibodies or antibody fragments, for example, Fab-fragments or their derivatives may be used as a ZK. The antibody or antibody fragments typically recognise structures on the target cell surface, eg. extracellular protein segments or extracellular carbohydrates that are membrane-residing or coupled to a protein, especially tumour antigens typical for the respective target cells, can be mentioned as epitopes.

In this context, lectins as ZK can be used for the binding to the target cell surface, as well as carbohydrate compounds in general. One of the two binding partners of the strept avidin-biotin-binding-reaction is also suitable as a ZK in a conjugate according to the invention. As mentioned previously, binding partners of a cell membrane-residing receptor or ligand, or a fragment or derivative of such a substance, are particularly preferred.

As unspecific ZK in a conjugate according to the invention, liposomes, hydrophobic anchors with the characteristic of settling inside the cell membrane, such as, for example, hydrocarbon chains, or lipid-like molecules, such as cholines, can be used, and especially preferred, aryl boronic acid derivatives or aryl boronic acid ester derivatives are presently disclosed. Here, the entire relevant teachings in the document DE 196 45 601 A1 are incorporated herein by reference. In particular, the aryl boronic acid derivatives described in claims 1 and 2 of the above-mentioned document, and the aryl boronic acid ester derivatives mentioned in claims 7, 8 and/or 9 of the same document, can be used as a ZK in a conjugate according to the invention. In particular, KZ is ZE-(B)n—, where ZE is a residue of a cell recognising unit. See, for example, DE 196 45 601, wherein n is 1; B is a boron group, and the cell recognising unit ZE is covalently linked either to the cross-linker group, thus being indirectly linked to the bicyclic anhydride group, which functions as a heterobifunctional cross-linker, or being directly linked to this bicyclic anhydride group.

For cell recognition of a conjugate according to the invention, the introduction of two cell recognising systems in the general formula (I) or (II), such as eg. two aryl boronic acids, proves to be particularly advantageous. Especially for coupling of a conjugate according to the invention to target cells, this ensures that a conjugate according to the invention, which masks an immunomodulator via a heterobifunctional cross-linker in an acid labile manner, will attach to the target cell surface over a particularly long period.

As acid labile heterobifunctional cross-linkers in the conjugate according to the invention, substances which are suitable are those which on the one hand have a stable bond to a ZK, but on the other hand are able to form an acid labile bond to another compound, eg. to an immunomodulator. This means that the bond of, for example, the immunomodulator to the cross-linker at a pH-value of eg. ≧7.4 is stable, whereas at a low pH-value, especially at a pH-value of ≦6.5, a compound coupled to the cross-linker is released. These substances will typically be bicyclic amides to which the immunomodulator is bound via an amino group in order to obtain a conjugate according to the invention. The present invention incorporates by reference all those cross-linkers in particular, which are also disclosed in DE 42 05 306 A1. Here, especially all those cross-linkers need to be mentioned, which have the characteristics of the compounds described in the claims 1, 2, and 3 of DE 42 05 306 A1.

Especially preferred, however, are bicyclic anhydrides as components of the conjugates according to the invention, as is disclosed in DE 43 26 273 A1. Thus, the complete disclosure of DE 43 26 273 A1 is included in the present application with regards to the bicyclic anhydrides as masking substances disclosed therein. Most of those cross-linkers, which are described by the characteristics of the claims 1, 2 and 3, can be used as cross-linkers for the immunomodulators on the one hand, and, on the other hand, for the ZK in a conjugate according to the invention.

In order to be able to couple the immunomodulator to a heterobifunctional, bicyclic cross-linker, which preferably masks the immunomodulator through its anhydride function, the immunomodulator should preferably contain a primary or secondary amino group. The hereby resulting amide bond with the cross-linker is labile and is preferably broken at pH-values of ≦6.8, more preferably at ≦6.5, and most preferably at ≦6.4.

Among the immunomodulators of a conjugate according to the invention, cytokines are preferred, especially the cytokines IL-1, IL-4, IL-6, IL-7, IL-2, INF-γ, TNF-αor GM-CSF. IL-2 is especially preferred because IL-2 is able to activate cytotoxic T-cells, as well as NK-cells. However, also chemokines, such as, for example, GROa, growth related on-cogene α; IL-8, Interleukin 8; MIP-2, macrophage inflammatory protein 2; IP-10, interferon-γ induceable protein 10; RANTES, regulated on activation, Normal T-cell expressed and secreted; MIP 1α, macrophage inflammatory protein 1α; MCP-1, monocyte chemo-attractant protein 1; and EOTAXIN or Ltn, lymphotactin, are suitable immunomodulators for the present invention. Also, all of the signal molecules referred to by the terms “monokines”, “lymphokines”, or “colony stimulating factors”, are preferred as immunomodulators in a conjugate according to the invention. Also, the factors known as “interferons” represent suitable immunomodulators. Furthermore, co-stimulating surface molecules of cells of the immune system or cells having a regulator function for the immune system also belong to the group of suitable immunomodulators. For example, the immunomodulator of a conjugate according to the invention can be the surface molecules CD40L, B7.1, CD80, CD70 and/or ICAM or other cell adhesion molecules. Finally, within the scope of the present invention, proteins which may be immunomodulators, include those which directly or indirectly influence the regulation of proteins (eg. the above-mentioned substances) involved in the immune action. These regulator proteins can be, for example, activators or inhibitors of DNA transcription or translation.

The immunomodulator can act by stimulating, preferably specifically on certain agents, eg. specifically by attacking tumour cells or attacking cells infected by bacteria or viruses, or they can act as inhibitors, eg., attenuate or inhibit excessive immune reactions, eg. in case of auto immune diseases.

All previously mentioned immunomodulators can be typically present in a conjugate according to the invention, in their physiological form. However, derivatives or fragments of physiological amino acid sequences can also be preferred, but it must be considered whether the physiological effectiveness of the derivatives or fragments used will remain intact. The fragments will typically be sequences truncated at the C- or N-terminal, but also non-terminal deletions are conceivable. Here, deletions between 1 and 50 amino acids are preferred, and deletions between 1 and 10 amino acids are particularly preferred. Sequences having one or more conservative amino acid substitutions are regarded as derivatives of immunomodulators, namely the exchange of a polar amino acid with another polar amino acid, for example, a hydrophobic or aromatic amino acid with another hydrophobic or aromatic amino acid.

However, immunomodulators which have been elongated with respect to the physiological sequences, especially N- or C-terminally elongated immunomodulators, are also disclosed according to the invention. The sequence elongations can have a functional character. For example, hydrides consisting of two or more immunomodulators of the previously mentioned kind, possibly separated by linker areas, can be fused and occur as an immunomodulator hybrid in a conjugate according to the invention. In this manner, several biological or immunological functions can be combined in one conjugate according to the invention such that an optimal effect of such a conjugate is achieved on a target cell, eg. with respect to its characteristics as part of a cancer vaccine.

Furthermore, derivatives can be produced from immunomodulators or immunomodulator hybrids by synthetic coupling of organic molecules. These can be labeling or diagnostic agents, eg. contrast dyes, which enable localisation of the vaccine against neoplastic disease in vivo by imaging procedures, or, eg. by means of fluorescence markers, to make the vaccine visible in cell tests in vitro by using relevant technical aids, eg. by means of fluorescence microscopes. One or more organic molecules can not only be coupled to the immunomodulator, but the molecule can also be coupled to the cross-linker and/or the ZK in the conjugate according to the invention. A conjugate according to the invention can also be derived with several, similar or different, organic molecules.

Possibly, one or more cytotoxic substances, especially chemotherapeutics, can also be used to derive an immunomodulator according to the invention by a covalent bond to a functional group of an immunomodulator according to the invention. According to the invention, especially those conjugates are disclosed which have two or more acid labile cross-linkers bound to only one ZK, where a cross-linker binds an immunomodulator, a derivative or fragment of same with the ZK, whereas one or more other cross-linkers, preferably different in their acid stability, conjugate one or more other compounds with the same ZK. Thus, different immunomodulators can each be combined onto a ZK via one cross-linker of the same or different type. However, in this manner, chemotherapeutics can be combined with immunomodulators in a conjugate molecule according to the invention, eg. the chemotherapeutic via an acid stable bond and the immunomodulator via an acid labile one. Such a conjugate according to the invention is especially advantageous when the conjugate is placed upon a neoplastic cell as an anti-neoplastic vaccine. Here, first of all the immunomodulator, bound to a more labile cross-linker, is released so that the vaccination reaction can proceed involving the neoplastic cell, the immunomodulator and the effector cell to be activated. Then, the chemotherapeutic is released with a delay, ideally after the immune reaction has taken place, in order to enable an additional cytotoxic reaction during the systemic application,—which, with high probability, will be aimed at a specific location against neoplastic cells due to their close vicinity. Instead of the chemotherapeutic, however, ligands such as the soluble Fas-ligand can also be released with a delay. Tthe soluble Fas-ligand causes an apoptotic reaction locally, preferably in the neoplastic tissue.

Another subject-matter of the present invention are methods for the production of conjugates of the type according to the invention. Production of the conjugates according to the invention assumes a conjugated 5- or 6-segment ring diene, substituted with one or two cell recognising units, ie. a furan, pyrrol, cyclopentadiene or conjugated cyclohexadiene. In a Diels-Alder reaction, maleic acid anhydride is added to this diene. The remaining double bond in the Diels-Alder product (unsaturated maleic acid anhydride adduct) can be reduced, if necessary, or be transformed with a peroxy acid or a nitrene in order to obtain the saturated, epoxidated or iminated maleic acid anhydride adduct. The saturated or unsaturated maleic acid anhydride adduct is then incubated with a bio-active substance, especially with an immunomodulator, which contains a primary or secondary amine forming the desired compound of formula I or II.

With the compounds or conjugates according to the invention it has become possible for the first time to link, by means of a labile bond, substances having an immunomodulating effect to a bicyclic linker-group having a cell recognising unit, thereby achieving a controlled release of the effective agents at the location of reaction. This new method opens up an unexpected and wide biochemical and medical range of applications, especially the use of such conjugates according to the invention for vaccination against neoplastic disease and for systemic therapy.

Conjugates of the type according to the invention, or compositions containing such conjugates, can also be administered systemically or via oral or parenteral application. It is preferred to apply the conjugates or the compositions containing such conjugates directly into the neoplastic lesion, provided that it is accessible. Thus, it is particularly preferred to inject compositions or conjugates according to the invention directly into a tumour, eg. in skin tumours, especially for treatment of a melanoma. The systemic administration is preferred if the conjugates according to the invention, or the compositions containing such conjugates, are to be used to attack neoplastic diseases having metastases, micro-metastases or individual dislocated tumour cells.

Another embodiment of the present invention is the use for treatment or for production of a medicament for treatment of autoimmune diseases, especially for the specific inhibition of the immunofunction, and for treatment of infection diseases, especially for specific stimulation of the immune system.

A further subject-matter of the present invention is an adduct for a target cell to which a conjugate according to the invention is bound. Conjugates according to the invention can be coupled to any target cells ex vivo or in vitro provided that the target cells have the binding partner of the ZK of the respective conjugate according to the invention. The binding to the target cell via the ZK can, eg., take place in a specific manner, as described above, or it can occur as binding to the cell surfaces via enzymes or via vicinal diols by means of the previously disclosed aryl boronic acids or aryl boronic acid esters.

In the case that an adduct according to the invention is to be used as a neoplastic disease vaccine, the target cell should preferably be a tumour antigen presenting cell. The cells, especially human cells and especially human cells of the immune system, with the use of gene-technology methods, can be transfected with DNA sequences for at least one membrane-residing antigen, preferably a tumour antigen. Typically, the transfected tumour antigens will be characteristic for the targeted neoplastic disease. It is preferred to transfect a cell, which should be the target cell in an adduct according to the invention, having two or three different antigens, preferably tumour antigens. The preferably human cells used for transfection will have been most preferably taken from the patient to be vaccinated (autologous cells) in order to exclude any rejection reactions of the patient after vaccination.

In another preferred embodiment of the present invention, the target cell in an adduct according to the invention can be a neoplastic cell of the patient to be vaccinated, which can be a target cell in an adduct according to the invention with or without the previously described transfection.

In a further preferred embodiment, not only will there be at least one conjugate of a class according to the invention bound to the target cell in an adduct according to the invention, but there are also conjugates of at least one other conjugate class (also conjugates of another structure) coupled to the same target cell. This means that in an adduct according to the invention a target cell can carry different conjugates according to the invention. They will in particular, be those combinations of different conjugates, each having different immunomodulators, which enhance the physiological immune response, ie. especially the activation of effector cells, by means of their biological synergism. Thus, for example, an adduct according to the invention can have conjugates according to the invention having a cytokine as immunomodulator on the one hand, but on the other hand, an adduct can also have conjugates having a co-stimulating immunomodulator on the target cell. Combinations of conjugates having different cytokines, eg. IFN-γ and IL-2, on a target cell to form an adduct according to the invention are also possible.

Another subject-matter of the present invention are compositions containing at least one adduct according to the invention. Compositions of the previously mentioned type can be based upon physiological saline solutions, and they may also contain other components, such as adjuvants.

Thus, within the scope of the present invention, vaccination methods are disclosed for the treatment of neoplastic disease, in particular including solid tumours. Here, a target cell, eg. a tumour cell, having tumour antigens at the membrane characteristic for the patient's tumour, which was previously removed from the respective tumour patient, is combined with conjugates of at least one conjugate class according to the invention to form a target cell conjugate adduct according to the invention. A tumour vaccine containing this adduct eg. in form of a suspension which may include other components, such as adjuvants, is then given to the patient in a further procedure step, typically eg. orally, but also parenterally, in particular via subcutaneous, intravenous or intramuscular injection. Administration of the adduct as a tumour vaccine can occur once or several times, preferably several times, with an interval of 7 to 14 days. Then, especially under acid conditions in the extracellular area, a controlled in vivo release of the immunomodulator from the tumour vaccine occurs after injection by means of the pH-labile predetermined break point at the amide bond, the bond of an amine to the heterobifunctional cross-linker. Thus, the immunomodulating effect of the immunomodulator only develops upon release under these conditions, i.e. especially in the tumour tissue where these conditions can be typically found.

The vaccination methods are preferred when adducts according to the invention, used as tumour vaccine either without any further components or as part of a composition having other components, receive radiation prior to the application in order to diminish the viability of the target cell, especially a neoplastic cell, present in the adduct. In this respect, adducts according to the invention, or compositions containing such adducts, eg. for use as vaccines against neoplastic disease, are most preferred if they have received radiation beforehand. Typically, radiotherapy is carried out at an intensity of 1000 to 15000 rad depending on the respective tumour entity.

Most preferred especially for vaccination methods, or for use of adducts in vaccinations against neoplastic disease, or for use of such adducts for production of a cancer vaccine, are such conjugates in the adduct having an unspecific ZK, especially an (aryl) boronic acid or an (aryl) boron acid ester. By means of the unspecific coupling agents, a conjugate according to the invention can be coupled to different target cells, eg. to target cells of different patients, without—as in the case with the specific coupling via receptor-ligand interaction—the disadvantage of having to accept repeated provision of respective suitable conjugates having a relevant ZK for the respective different membrane-residing binding partner at different (patient) target cells. Thus, in this manner, different target cells can be marked with the same conjugate. As a result, by means of these identical conjugates according to the invention and their ability to bond unspecifically, adducts suitable for vaccination can be provided, each being different (with regards to the target cells).

The previously mentioned methods or compositions according to the invention, or use of adducts according to the invention, containing an adduct according to the invention, for production of a vaccine for treatment of neoplastic diseases are especially suitable for treatment of leukemia diseases, especially acute lymphoblastic leukemia and myeloic leukemia in children, but also for treatment of acute leukemia in adults, for treatment of non-Hodgkin lymphomas and the plasmozyton, as well as the Hodgkin lymphomas. In addition, they have also proved to be useable for the treatment of skin tumours, especially melanomas and squamous epithelial carcinoma, as well as for the treatment of solid tumours of the lung, intestine, especially the colon carcinoma, the stomach, the kidney and the pancreas.

However, those substituted heterofunctional substituted cross-linkers can also be regarded as conjugates according to the invention, to which a specific or unspecific cell recognition unit is coupled, especially an unspecific ZK, in particular an aryl boron acid or an aryl boron acid ester, where all previously disclosed variants are possible, as well as a bio-active substance not having any immunomodulating effect. Most preferred are bio-active substances which are suitable for treatment of neoplastic disease. Some of the potential bio-active substances are listed below as examples. This list, however, is not comprehensive, and is not intended to limit the invention.

Thus, bio-active substances include especially growth factors such as EGF, GCSF, GGF, GMF, GMA, GMCF, IGF, NGF, PDGF, PD-ECGF, TGF or VDGF, growth factors of the haemotopoetic system, such as eg. MDGF, SCF, FLT-3L, especially also other angiogenesis factors, or also anti-angiogenesis factors, such as eg. endostatin or angiostatin; cell differentiation factors, such as eg. BMP, TGF, VEGF, VDGF; also higher level hormones in the control cascade, eg. hormones secreted by the hypothalamus or hypophysis, such as eg. releasing hormones, especially GHRH or TRH, or ACTH, somatotropin or LH. Furthermore, factors have to be identified as bio-active substances, which are involved in the apoptosis, eg. FasL, possibly also NFKB. Especially preferred as bio-active substances are also mimetics of the previously mentioned hormones, especially when they are modified in such a manner, eg. synthetically, or in the case of peptide or protein hormones by sequence changes such as substitutions, insertions and/or deletions, so that while they can bind to their physiological receptors, they then cannot trigger the physiological signal. In this case, the mimetics of the bio-active substances in the conjugate according to the invention block the respective receptors and are thus in vivo preferably competitive inhibitors of the genuine ligands.

The bio-active substances, however, can also be the receptors, especially the soluble forms of membrane-residing receptors of the previously mentioned ligands. If these receptors or receptor fragments, especially the extracellular domains of the receptors, are provided as bio-active substances in a conjugate according to the invention, they can be released in a suitable, physiological environment, catching the ligands, which occur there, in a competitive binding reaction. Thus, the physiological signal transduction at the cell membrane is attenuated or, ideally, inhibited.

In yet another embodiment of the invention, chemotherapeutics, such as cytotoxins, eg. cis-Platin, Carboplatin, Procarbazin, Mitoxanthron, Doxorubicin, Zorubicin, Epirubicon, Melphalan, Nor-N-lost, Mitomycon C, or Bleomycin, radiochemotherapeutics, or enzyme inhibitors can be regarded as bio-active substances which can occur in the conjugate; finally also antibiotics, such as penicillins, cephalosporines, streptomycines, and many more. In general, native or synthetic peptides having inhibitor or activator function for receptors or ligands, enzymes or proteins of the signal transduction channel may also be suitable.

In addition, factors influencing the signal transduction or the cell differentiation in the broadest sense can be regarded as bio-active substances. In addition, bio-active substances can also be those substances which influence the DNA or RNA of cells, especially tumour cells. Thus, for example, anti-sense DNA or RNA, or ribozymes can be coupled to conjugates according to the invention, which recognise, bind and/or cut the DNA or RNA sequences specific for the respective tumours, typically regulating the hyperproliferation of the cells. Since such ribozymes or anti-sense DNA or RNA preferably have an intracellular effect, the conjugate should be designed in a manner that the anti-sense molecule or the ribozyme can penetrate into the tumour cells.

Typically, there are two possibilities to ensure absorption of a bio-active substance into the intracellular area. If a conjugate according to the invention having a bio-active substance, or a compound containing such a conjugate, is administered systemically, incorporation by means of a suitable choice of a ZK, as described above, is ensured. Here, the ZK binds to a membrane-residing binding partner onto the surface the target cell; whereas the binding partner, on the cell surface after binding the conjugate, has the physiological characteristic to be internalised.

If, however, an adduct is produced ex vivo from the target cell and a conjugate and then administered to the patient, the incorporation of the bio-active substance in the adduct is solved according to the invention in that the bio-active substance forms two covalent bonds: first, an acid labile bond according to the invention to a heterobifunctional cross-linker, second another bond directly to another ZK. This additional ZK is chosen in such a manner that it has a membrane-residing surface molecule of eg. tumour cells as a binding partner, where the surface molecule, after binding its binding partner, has internalisation ability. Thus, for example, an adduct designed in that way, after separating the acid labile bond between cross-linker and bio-active substance, will release the bio-active substance in the extracellular space. This bio-active substance, localised in the extracellular space, is, however, bonded covalently with another ZK. Via this ZK, the bio-active substance can bind to the respective binding partner on the target cell surface, typically a neoplastic cell, and it will then be absorbed into the intracellular space due to the characteristics of the binding partner. In this way, bio-active substances with intracellular effect, eg. ribozymes or anti-sense molecules, can be infiltrated into cells after adducts according to the invention were administered.

Thus, it is possible, to apply conjugates having a bio-active component in the above sense, as a medicament, for use as a medicament, or for use in the production of medicaments for treatment of neoplastic diseases. Especially useful bioactive components include anti-sense molecules, or ribozymes, the previously mentioned chemotherapeutics, or the previously mentioned competitive mimetics of growth factors, antibiotics for bacterial infections, previously mentioned growth factors for speeding up the healing process of wounds and fractures, angiogenesis factors, for vessel development and for genesis of tissues, eg. for the replacement of organs. Another bio-active component suitable for use in the invention is a previously mentioned anti-angiogenesis factor for prevention of vessel development and also for attacking neoplastic tissue.

For conjugates having a bio-active substance, where the bio-active substance does not have immunomodulating effects, the entire previous disclosure regarding immunomodulating effective conjugates according to the invention and their preferred embodiments applies accordingly. Reference is also made to the disclosure regarding compositions having such conjugates, or regarding adducts from conjugates and target cells, provided that the conjugates contain bio-active substances in the above sense.

The present invention is further explained in detail by means of the following embodiments:

Embodiment 1: Synthesis of a conjugate according to the invention having a heterobifunctional cross-linker, a cell recognising unit (ZK) and an immunomodulator of the general formula (II):

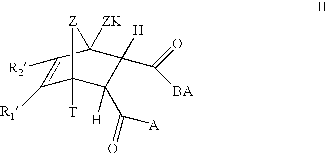

where: R′₁=R′₂=T=H; Z=O; ZK=B(OH)₂; A=OH; BA=IL-2

a) Synthesis of Maleic Acid Anhydride Adduct of 2-Furyl Boronic Acid

10.000 g (0.084 Mol) 2-furyl boronic acid (Aldrich) are dissolved in 100 ml diethyl ether/THF (1:1). Then, 8.064 g (0.084 Mol) maleic acid anhydride is added. After 20 hours, the reaction solution is carefully distilled under reduced pressure. Thus, 15 g (83.3%) of the Diels-Alder adduct is obtained.

IR (KBr)=1895 cm⁻¹.

b) Synthesis of the Interleukin Conjugate

100 μg of Interleukin-2 (IL-2, Calbiochem) are incubated for a period of 30 minutes at room temperature in 20 μl of 10 mM phosphate, pH 7.5, with 10 μg of the Diels-Alder adduct produced in a). Subsequently, the reaction solution is filtered through an amino doted micro-cellulose filter into a 1% human serum albumin (HAS) solution in order to remove excessive reactive anhydride material. It is then divided into portions and deep-frozen.

Embodiment 2: Synthesis of a conjugate according to the invention having a heterobifuctional cross-linker, two cell recognising units and an immunomodulator of the general formula (II):

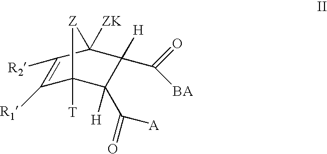

Where: R′₁R′₂=H; Z B(OH)₂; T=CH₂—N(C(O)CH₃) (C₆H₄)-meta-B(OH)₂; A=OH; BA=IL-2

a) Synthesis of the Starting Compound 5-[N-(3-dihydroxyborylphenyl)-N-acetylamonomethyl]-2-furyl Boronic Acid

5 g of 2-furyl-5-formyl boronic acid (0.035 Mol) (Aldrich) are mixed with 6.22 g (0.035) of 3-aminophenyl boronic acid (Aldrich) and an equivalent of triethylamine and then condensed in the melter. Then, the base product is suspended in 10 ml of water and 10 ml of methanol and mixed with an excess of sodium cyano boron hydride and stirred for two days at room temperature. Then, the pH-value is set to pH 8, and the base product is extracted with dichloromethane. The solvents are distilled, and the residue is treated over night with pyridine/acetanhydride. Subsequently, the solvent is distilled, the residue is absorbed with water and extracted with ethylacetate. After re-crystallisation, 1.8 g (17%) of the desired compound is obtained.

b) Synthesis of the Maleic Acid Anhydride Adduct of 5-[N-(3-dihydroxyborylphenyl)-N-acetylaminol]-2-furyl Boronic Acid.

1.000 g (0.003 mol) of the bisboronic acid produced in step a) are dissolved in 10 ml of diethylether/THF (1:1). Then, 0.324 (0.003 mol) maleic acid anhydride are added. It is stirred for a period of 20 hours, then the reaction solution is carefully distilled under decreased pressure. Thus, 1.3 g (95%) of the Diels-Alder product having two cell recognising systems ZK is obtained.

IR (KBr)=1898 cm⁻¹.

c. Synthesis of the Interleukin-2-Conjugate.

For a period of 30 minutes, at room temperature, 100 μg Interleukin-2 (Calbiochem) are incubated in 20 μl of 10 mM phosphate, pH-value 7.5, with 10 μg of the Diels-Alder product produced in step b). Then, the reaction solution is filtered via an amino-doted micro-cellulose filter into a 1% HFA solution (in order to remove excess anhydride reagents), and it is subsequently divided into portions and deep-frozen.

Embodiment 3: Production of an adduct according to the invention from a conjugate according to the invention, based on embodiment 1 or 2 and a target cell. Based on the embodiments 1 or 2, conjugates according to the invention were bound to (i) murine or to (ii) human neoplastic cells of neuronal or haematopeotic origin. Here, an anhydride was first transformed with an aryl boron acid as a ZK and a cell marker (fluorescein) or —in a parallel approach—an immunomodulator (IL-2) into heterobifunctional conjugates.

To prove the cell binding, 100 μM of the fluorescein marked conjugate were incubated with 2×10⁵ target cells in a volume of 1 ml in a 15 ml test tube. After incubation was started, the cell binding was determined at different intervals in a flow cytometer by means of the intensity of the measured fluorescence.

The binding of the molecule to the target cells occurred within few minutes, where by means of fluorescence marking of the aryl boron acid as a ZK it could be established that, after 5 minutes, all cells (100%) were already marked. This binding of the conjugates according to the invention to the target cells in all four approaches (murine tumour cells of neuronal origin, murine cells of haematopoetic origin, human tumour cells of neuronal origin, and human tunour cells of haematopoetic origin) remained stable at a temperature of 4° C., and at a physiological temperature of 37° C.

In the above-mentioned four cell cultures of the adducts according to the invention, no changed cell proliferation could be observed with respect to the control cell cultures (the above-mentioned tumour cells without coupling of a conjugate according to the invention). In addition, no apopoetic processes, no changed cell adhesion, and also, no changed antigen presentation was established for the adducts according to the invention. The results were obtained from relevant standard tests known to those skilled in the art, e.g. examinations regarding the apoptosis using the propidiumiodide test including subsequent flow cytometrics or including antibodies dyeing, where the antibody recognise T-cells, and from subsequent detection by means of flow cytometrics.

Embodiment 4: Release of the immunomodulator in vitro The adducts according to the invention from conjugate and tumour target cell, obtained according to embodiment 3, were cultured. During the initial 12 hours of cultivation, in two-hour intervals each time, surplus cells were taken from the cell culture. Then, that process was carried out in 12-hour intervals. The concentration of released immunomodulators, ie. in this case IL-2, contained in these surplus cells was determined by means of ELISA (enzyme linked immuno assay).

Here, it was established that 106 cells released ≧500 pg of IL-2 in a period of 24 h, where a half-life period of the conjugate of 100 h had been set.

Embodiment 5: Tumour vaccination with an adduct according to the invention based on embodiment 3.

Each of the adducts obtained according to embodiment 3 having murine neoplatic cells of haemopoetic origin (leukemia cells) were tested as neoplastic disease vaccines in mice. The tumour model system was established by injecting mice subcutaneously with leukemia cells. The growth of the tumour in the subcutaneous tissue before and after vaccination was observed by measurements taken in three-day intervals. Three or 10 days after subcutaneous injection of the leukemia cells, the mice, which had been prepared in such a manner, were treated with the vaccine (see embodiment 3). Here, the neoplastic disease vaccine was injected subcutaneously, where the leukemia cells contained in the vaccine had previously been treated with radiation. As a control test, leukemia cells, which were not coupled to a conjugate according to the invention, and which were also treated with radiation, were applied to the mice in the same manner as the neoplastic disease vaccine.

Here, with the majority of the mice, it showed that a suppression of the neoplastic growth could be established, which resulted in a clearly increased long-term survival rate. The immune system response achieved by the vaccination is conveyed through the T-cells, which could be proven in vivo by means of depletions of immunological effector cell populations.

### Equivalents

While this invention has been particularly shown and described with references to certain embodiments thereof, it will be understood by those skilled in the art that various changes in form and details may be made therein without departing from the spirit and scope of the invention encompassed by the appended claims. Such equivalents are intended to be encompassed in the scope of the following claims.

1. A compound of Formula I or II: 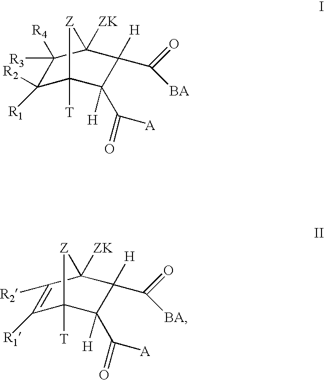
wherein BA is a bio-active group attached to carbonyl by an acid labile bond;
ZK is a cell surface binding group or a cell recognising group;
R₂ and R₄ are hydrogen or together form —O— or —NR₁₀—, wherein R₁₀ is a linear chain or branched alkyl of 1 to 6 carbon atoms;
R₁, R₃, R′₁ and R′₂ are each chosen independently from the group consisting of hydrogen, alkyl, and substituted alkyl; or
R₁ and R₃ together, or R′₁ and R′₂ together form a 5 to 7 segment unsaturated heterocycle of 1 to 3 heteroatoms selected from N—, S—, and O; or R₁ and R₃ together, or R′₁ and R′₂ together form a 4 to 6 segment carbocycle;
Z is chosen from the group consisting of oxygen, sulfur, —NR₅—, —CR₆R₇—, and CR₆R₇—CR₈R₉—, wherein R₅ is H or C₁₋₈-alkyl, and R₆, R₇, R₈ and R₉ are each independently H or C₁₋₆-alkyl;
A is hydroxy; and
T is chosen from the group consisting of a cell surface binding group, a cell recognising group, hydrogen, alkyl, and substituted alkyl:

2. A compound according to claim 1, of Formula I or II: 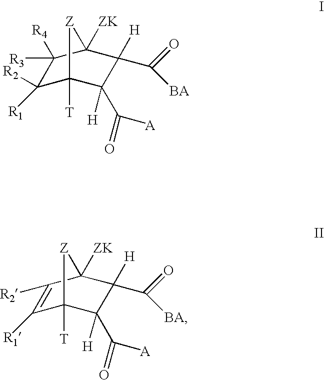
wherein BA is an immunomodulator group or an anti-neoplastic group attached to carbonyl by an acid labile bond;
ZK is a cell surface binding group or a cell recognising group;
R₂ and R₄ are hydrogen or together form —O— or —NR₁₀—, wherein R₁₀ is a linear chain or branched alkyl of 1 to 6 carbon atoms;
R₁, R₃, R′₁ and R′₂ are each chosen independently from the group consisting of hydrogen, alkyl, and substituted alkyl; or
R₁ and R₃ together, or R′₁ and R′₂ together form a 5 to 7 segment unsaturated heterocycle of 1 to 3 heteroatoms selected from N—, S—, and O; or
R₁ and R₃ together, or R′₁ and R′₂ together form a 4 to 6 segment carbocycle;
Z is chosen from the group consisting of oxygen, sulfur, —NR₅—, —CR₆R₇—; and
—CR₆R₇—CR₈R₉—, wherein R₅ is H or C₁₋₈-alkyl, and R₆, R₇, R₈ and R₉ are each independently H or C₁₋₆-alkyl;
A is hydroxy; and
T is chosen from the group consisting of a cell surface binding group, a cell recognising group, hydrogen, alkyl, and substituted alkyl.

3. The compound according to claim 2, wherein BA is an anti-neoplastic group.

4. The compound according to claim 2, wherein BA is an immunomodulator.

5. The compound according to claim 4, wherein the immunomodulator is a primary amine or a secondary amine.

6. The compound according to claim 4, wherein the immunomodulator is a protein or a protein segment.

7. The compound according to claim 4, of Formula II, wherein R₁, R₂ and T are H;
Z is O; and
ZK is —B(OH)₂.

8. The compound according to claim 3, of Formula II, wherein R₁, R₂ and T are H;
Z is O; and
ZK is —B(OH)2.

9. The compound according to claim 4, of Formula II, wherein R₁ and R₂ are H;
T is CH₂—N(C(O)-meta-C₆H₄—B(OH)₂, Z is O; and
ZK is —B(OH)₂.

10. The compound according to claim 3, of Formula II, wherein R₁ and R₂ are H;
T is —CH₂—N(C(O)-meta-C₆H₄—B(OH)₂,
Z is O; and
ZK is —B(OH)₂.

11. The compound according to claim 4, wherein the immunomodulator is chosen from the group consisting of cytokine, chemokine, monokine, lymphokine, interferon, a cell surface protein, and a protein involved in activating at least one immunomodulator.

12. The compound according to claim 11, wherein the protein involved in activating the immunomodulator is a transcription factor or a derivative of a transcription factor.

13. The compound according to claim 4, wherein the immunomodulator is an interleukin.

14. The compound according to claim 13, wherein the interleukin is IL-2.

15. The compound according to claim 2, wherein ZK is chosen from the group consisting of an antibody or derivative or fragment thereof, a lectin or derivative or fragment thereof, a carbohydrate compound or derivative or fragment thereof, a binding partner of a membrane-residing receptor or ligand, or derivative or fragment of the binding partner, a liposome, and a cell-recognising aryl boronic acid.

16. The compound according to claim 1, wherein the bio-active group is chosen from the group consisting of a growth factor, an angiogenesis factor, an anti-angiogenesis factor, a signal transinduction inhibitor, an enzyme inhibitor, an apoptosis inducing compound, an antibiotic, a chemotherapeutic compound, a radiochemotherapeutic molecule, and a radiomarker molecule.

17. The compound according to claim 2, wherein at least one additional substance is covalently bonded to the compound.

18. The compound according to claim 17, wherein the one additional substance is bonded to BA.

19. The compound according to claim 18, wherein the additional substance is a bio-active compound.

20. The compound according to claim 19, wherein the bio-active compound is chosen from the group consisting of a group that is the same as BA, an immunomodulator or an anti-neoplastic group that is different from BA, an angiogenesis factor, an anti-angiogenesis factor, a signal transinduction inhibitor, an enzyme inhibitor, an apoptosis-inducing compound, an antibiotic, a non-radioactive chemotherapeutic substance, a radiochemotherapeutic substance, and a radiomarker molecule.

21. The compound according to claim 20, wherein the radiomarker is a fluorescence marker.

22. The compound according to claim 1, wherein the compound is multimerised.

23. The compound according to claim 22, wherein the compound is di-, tri-, tetra- or pentamerised.

24. A pharmaceutical composition comprising a pharmaceutically acceptable carrier and a compound according to claim 1.

25. A method for treating a neoplastic disease in a patient in need thereof comprising administering to said patient a therapeutically effective amount of a compound according to claim 1.

26. The method of claim 25, wherein the disease that is treated is leukemia.

27. A method for treating an autoimmune disease or an infectious disease in a patient in need thereof, or inhibiting the onset of symptoms of the autoimmune disease or the infectious disease, comprising administering to said patient a therapeutically effective amount of a compound according to claim 1.

28. A method for producing a compound according to claim 1 of the general formula I or II: 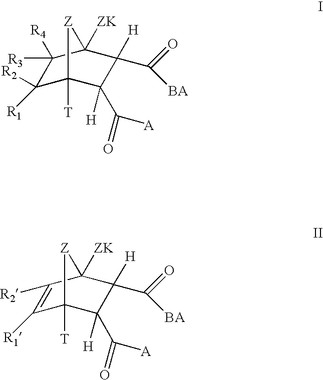
wherein
R₁, R₃, R′₁, R′₂, A, T, Z, ZK and BA are as defined in claim 1; and
R₂ and R₄ are H, the method comprising the steps of:
a) reacting a diene of formula III: 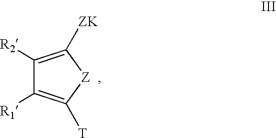
wherein R′₁, R′₂, T, Z and ZK are as defined in claim 1, with maleic acid anhydride, thereby forming a product of formula IV: 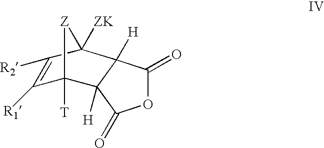
wherein R′₁, R′₂, T, Z and ZK are as defined in claim 1;
(b) optionally reducing the product of formula IV, thereby forming a product of formula V: 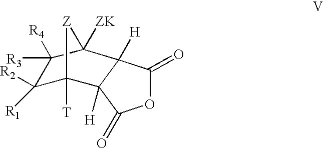
wherein R₁, R₃, T, Z and ZK are as defined in claim 1; and R₂ and R₄ are H; and
c) incubating the product of formulas IV or V with a bio-specific compound containing a primary or secondary amine group, thereby obtaining a product of formula I or II.

29. An adduct of at least one compound according to claim 1 and a cell, wherein the compound is coupled specifically or unspecifically to the cell via ZK.

30. The adduct according to claim 29, wherein the cell is a neoplastic cell or a human immune cell.

31. The adduct according to claim 30, wherein the human immune cell is chosen from the group consisting of a dendritic cell, a B-lymphocyte, and a T-lymphocyte.

32. The adduct according to claim 29, wherein at least two compounds of at least two different classes are coupled to the cell.

33. The adduct according to claim 32, wherein at least two compounds coupled to the cell have different immunomodulators.

34. An adduct according to claim 29, comprising at least two compounds and at least one additional component coupled to the cell.

35. An adduct according to claim 34, wherein the compounds coupled to the cell are the same or different.

36. A method for treating a neoplastic disease in a patient in need thereof comprising administering to said patient a therapeutically effective amount of a composition comprising an adduct according to claim 29.

37. A method for treating a neoplastic disease in a patient in need thereof comprising administering to said patient a therapeutically effective amount of a composition comprising an adduct according to claim 30.
